(19) United States
(12) Patent Application Publication
(10) Pub. No.: US 2004/0214193 A1
(43) Pub. Date: Oct. 28, 2004

(54) Method for assessing recording in vitro and in vivo

(75) Inventors: Laurence C. Eisenlohr, Merion Station, PA (US); Michael T. Howard, Salt Lake City, UT (US)

Correspondence Address: David S. Resnick, NIXON PEABODY LLP, 100 Summer Street, Boston, MA, 02110, US

(73) Assignees: Thomas Jefferson University, Philadelphia, PA (US); University of Utah Research Foundation, Salt Lake City, UT (US)

(21) Appl. No.: 10/627,588

(22) Filed: Jul. 28, 2003

Related U.S. Application Data
(63) Continuation of application No. 09/981,393, filed on Oct. 16, 2001, now abandoned.
(60) Provisional application No. 60/241,071, filed on Oct. 17, 2000.

(51) Int. Cl. C12Q 1/68; C12P 19/34

(52) U.S. Cl. 435/6; 435/91.2

(57) ABSTRACT

Recoding of the genetic code, through +1 frameshifting, −1 frameshifting or stop codon readthrough, will alter the protein that is translated from that gene. Current systems that quantify recoding events have limited sensitivity, and can only be used in cell extracts or tissue culture. A novel method for detecting a recoding event is described that uses the sensitivity and specificity of CD8+ T-cells for measuring recoding, both in vivo and in vitro. This enhanced sensitivity allows for the identification of compounds that are used to regulate recoding, and therefore protein translation.

### CROSS REFERENCE TO RELATED APPLICATIONS

This application claims priority under 35 U.S.C.§119 based upon U.S. Provisional Patent Application No. 60/241,071 filed Oct. 17, 2000.

### FIELD OF THE INVENTION

The present invention relates to the field of molecular biology, and more particularly to a method for measuring the recoding of protein translation and the use of this method for testing the efficacy of compounds in their ability to influence the recoding of protein translation.

### BACKGROUND OF THE INVENTION

Protein translation occurs with a high degree of fidelity. In general the rules of translational decoding are universal, however some genes are able to break the rules of decoding in response to specific regulatory elements carried within the RNA message (Gesteland et al., Science 257, 1640-1, 1992). This alternate reading of the genetic code is referred to as recoding. Recoding comes in at least four categories: +1 frameshifting, −1 frameshifting, stop codon readthrough or redefinition, and one example of a translational bypass of 50 nucleotides in T4 gene 60 (Gesteland and Atkins, Annu Rev Biochem 65, 741-68, 1996). These types of events are to be clearly distinguished from simple errors that occur at a very low frequency under normal conditions.

Many viruses use recoding as a means for regulating gene expression. For example, the retrovirus HIV-1 uses a −1 frameshift event to regulatete relative levels of expression of the g -pol protein required for viral replication (FIG. 1). The genes for gag and pol are contained on a single mRNA and translation of pol only occurs if the ribosome shifts into the −1 frame at the end of the gag gene (Jacks et al., Nature 331, 280-3, 1988). This frameshift requires a specific frameshifting motif XXXYYYN found in many examples of −1 frameshifting where, for HIV, X and Y are U and N is G. This “slippery” motif is followed by an RNA stem loop structure that serves to modulate the frequency of frameshifting (Jacks et al., Nature 331,280-3, 1988).

The only mammalian cellular gene known to undergo +1 frameshifting is Ornithine Decarboxylase antizyme (antizyme). Antizyme is a critical regulatory protein involved in polyamine homeostasis within the cell (Hayashi et al., Trends Biochem Sci 21, 27-30, 1996). The expression of antizyme is regulated by a +1 translational frameshift (Ivanov et al., Genomics 52, 119-29, 1998; Ivaylo et al., J.Biol. Chem., 1999; Matsufuji et al., Cell 80, 51-60, 1995; Rom and Kahana, Proc Natl Acad Sci USA 91, 3959-63, 1994). Each antizyme gene contains two open reading frames with the second downstream ORF in the +1 reading frame relative to the upstream ORF. It has been demonstrated that frameshifting of antizyme occurs at a specific site that is determined by an adjacent stop codon in the 0 frame, as well as RNA sequences 5′ and an RNA pseudoknot 3′ of the shift site (Matsufuji et al., Cell 80, 51-60, 1995) (see FIG. 1).

Stop codon readthrough occurs when a standard stop codon is decoded by a tRNA as a result of signals in the messenger RNA. Examples of this include the MuLV gag-pol gene expression and a number of nuclear encoded selenoproteins in mammals (Gesteland and Atkins, Annu Rev Biochem 65, 741-68, 1996). In the case of MuLV, the pol protein is expressed as a result of ribosome readthrough of the gag gene stop codon stimulated by a downstream RNA pseudoknot (Wills et al., Proc Natl Acad Sci USA 88, 6991-5, 1991).

Selenoproteins are a special case in which a novel tRNA aminoacylated with selenocysteine is used to decode a normal stop codon when a special RNA signal is located in the 3 UTR of a gene erry and Larsen, Biochem Soc Trans 21, 827-32, 1993). The selenocysteine amino acid, which is incorporated into the protein, is often a key residue within the active site of that protein.

In addition to these examples of recoding, it has been demonstrated that the aminoglycosides, including gentamicin, G418, and paramomycin, can induce ribosomes to undergo stop codon readthrough (FIG. 1) independently of a programmed RNA signal at relatively high frequencies (1-20%) (Mankin and Liebman, nat Genet 23, 8-10, 1999).

Drug Design in the Treatment of Infectious Agents

Many viruses and retroviruses use recoding as a way of controlling levels of gene expression. It is generally believed that the level at which these genes are expressed has been fine tuned by evolution and selective pressures to be at an optimal level for the viral life cycle. Any deviation from this frequency will inhibit the propagation of any virus that uses recoding, including for example, but not limited to, HIV (Irvine et al., N Z Med J. 111, 222-4, 1998), MMTV, HTLV-1, HTLV-2, SIV, MuLV and RSV. Antisense technologies or chemical compounds which target recoding will have potent antiviral activities due to their effect on viral gene expression. The present invention identifies the efficacy of compounds in their ability to recode a viral protein, thereby inhibiting viral gene expression and subsequently viral proliferation.

Drug Design in the Treatment of Cancer

Mammalian antizyme, whose expression is regulated by a +1 frameshift event, is a critical component in maintaining intracellular polyamine within an optimal range (Hayashi et al.,Trends Biochem Sci 21, 27-30, 1996). Elevated polyamine levels are associated with cellular proliferation and transformation, whereas, polyamine depletion is known to inhibit cellular growth and extreme depletion results in cell death (Pegg, Cancer Res 48, 759-74, 1988). Although the exact mechanism by which polyamines exert their effects on cellular growth and proliferation is not known, it is clear that the intracellular levels of polyamines are highly regulated by a complex mechanism involving antizyme and recoding.

Ornithine decarboxylase (ODC) is the first and rate limiting enzyme in the formation of the polyamines putrescine, spermidine and spermine (Tabor and Tabor, Annu Rev Biochem 53, 749-90, 1984). The intracellular levels of these polyamines are tightly regulated by a feedback mechanism which controls not only the levels of ODC but also polyamine transport into the cell. This feedback mechanism is mediated by antizyme (Hayashi et al., Trends Biochem Sci 21, 27-30, 1996). Antizyme forms a direct complex with ODC resulting in inhibition (Fong et al., Biochim Biophys Acta 428, 456-65, 1976; Heller et al., Proc Natl Acad Sci USA 73, 1976) and increased degradation of ODC (Bercovich and Kahana, Eur J Biochem, 205-10, 1993; Li and Coffino, Mol Cell Biol 13, 2377-83. 1993; Murakami et al., Nature 360, 597-9, 1992; Murakami et al., J Biol Chem 267, 13138-12, 1992). In addition, antizyme is responsible for inhibiting polyamine transport into the cell(Mitchell et al., Biochem J 299, 19-22, 1994; Suzuki et al., Proc Natl Acad Sci USA 91, 8930-4, 1994). Thus, a regulatory loop is defined by the ability of the polyamines to increase antizyme expression (by stimulating recoding) resulting in the shutdown of polyamine synthesis and transport.

Recent efforts in the development of anticancer chemotherapeutics have applied the strategy of targeting antizyme recoding as a means to lower polyamine levels and inhibit cellular proliferation (Marton and Pegg, Annu Rev Pharacol Toxicol 35, 55-91, 1995). Compounds such as the natural polyamine agmatine and other polyamine analogues are capable of stimulating antizyme expression via their effect on +1 frameshifting and result in lowered polyamine levels (Marton and Pegg, Annu Rev Pharacol Toxicol 35, 55-91,995; Satriano et al., J Biol Chem 273, 15313-6,1998). However, they do not substitute for the essential cellular proliferation functions of the polyamines and consequently result in growth inhibition of transformed cell lines (Satriano et al., J Biol Chem 273, 15313-6, 1998). The present invention identifies the efficacy of compounds in their ability to recode a gene, thereby influencing proliferation. Genes that are recoded include, but are not limited to, the mammalian antizyme. The identification of novel compounds that influence the proliferative capacity of a cell are useful in the treatment of cancers (where there is excessive proliferation) and degenerative diseases (where there is excessive cell death).

Drug Design in the Treatment of Genetic Diseases:

A large number of human genetic diseases result from point mutations that result in premature termination of protein synthesis of the mutant gene. It has been estimated that between 5-15% of all patients that suffer from Duschenne Muscular Dystrophy carry point mutations that result in a premature stop codon in the dystrophin gene (Barton-Davis et al., J Biol Chem 273, 15313-6,, 1999). This is probably an accurate estimate for the occurrence of this type of mutation in other diseases as well. Studies as early as 1979 indicated that treatment of eukaryotic cells with aminoglycosides can result in stop codon readthrough at these types of mutations (Palmer et al., Nature 277, 148-50, 1979; Singh et al., Nature 277, 146-8, 1979). The possibility that these drugs could be used to partially restore normal protein levels in patients carrying such a mutation has recently been raised (Mankin and Liebman, Nat Genet 23, 8-10, 1999). Aminoglycoside treatment of the mdx mouse carrying a premature stop codon within the dystrophin gene resulted in approximately 20% normal dystrophin expression and partial reduction of disease symptoms in these animals(Barton-Davis et al., J Clin Invest 104, 375-81, 1999). Similar results have been obtained in cellular models of cystic fibrosis (Bedwell et al., Nat Med 3, 205-10, 1997; Howard et al., Nat Med 2, 222-4, 1996). These are exciting results and will surely lead to active research in developing more effective drugs for suppressing premature stop codons.

The present invention identifies the efficacy of compounds in their ability to cause translational readthrough of stop codons or translational frameshifting, thereby restoring normal protein levels in patients carrying a premature stop codon or frameshift mutation, respectively. By restoring normal protein levels in patients carry such premature stop codons, disease symptoms are alleviated.

State of the Art in Measuring Recoding:

The current state of the art for measuring examples of recoding and stop codon suppression involves the use of enzymatic reporter genes. In these experiments, recoding sequences or stop codons are positioned upstream of a reporter gene such that when recoding occurs the reporter gene will be expressed. These plasmids are then transiently or stabily transfected into eukaryotic cells in tissue culture or transcribed and translated in cell free extracts. The amounts of expression from the reporter genes relative to controls are then used to deduce the frequency of recoding or stop codon suppression. Disadvantages include: 1) the large size of the reporter genes which may carry sequences that effect recoding, 2) limited sensitivity, and 3) limitation of the assay to cell extracts or tissue culture cells.

The state of the art techniques to test translational regulation of gene expression in mice relies on the production of transgenic mice. These mice must be generated for each sequence being tested using conventional reporter genes. This is an extremely time consuming and resource consuming process. The invention disclosed herein allows for individual clones to be produced in E. coli using traditional cloning techniques. Tens to hundreds of sequences are efficiently analyzed by the method of the present invention in non-transgenic mice. The extreme sensitivity of the mouse immune system allows translational gene regulation to be measured effectively and efficiently.

The present invention fulfills a long sought need for a simple system. The system of the present invention relies on a smaller reporter sequence, increases sensitivity, and is used in an animal model to determine the in vivo efficacy of a test compound in recoding a gene. The invention disclosed herein allows for the screening of compounds that influence recoding or stop codon suppression for the purpose of treating viral infections, cancer and genetic diseases.

### DEFINITIONS

“peptide antigen” means “epitope”

### ABBREVIATIONS

AZ, ornithine decarboxylase antizyme;

BSS/BSA, balanced salt solution with 0.1% bovine serum albumin;

HSV, herpes simplex virus;

NP, nucleoprotein;

NP₅₀₋₅₇, an H-2K^{k}-restricted epitope within NP;

NP₁₄₇₋₁₅₅ an H-2K^{d}-restricted epitope within NP;

NP₃₆₆₋₃₇₄, an H-2D^{b}-restricted epitope within NP;

ODC, orinithine decarboxylase;

ORF, open reading frame;

Ova₂₅₇₋₂₆₄, and H-2K^{b}-restricted epitope within ovalbumin;

RF, reading frame;

RF0, the conventional open reading frame;

RF−1, the −1 reading frame;

RF+1, the +1 reading frame;

rVV, recombinant vaccinia virus;

T_{CD8+}, CD₈₊ T cell;

TK, thymidine kinase;

VV, vaccinia virus

### DESCRIPTON OF THE DRAWINGS

FIG. 1. Schematic representation of recoding events for protein translation. The top panel shows a −1 frameshifting event, the center panel shows a +1 frameshifting event and the bottom panel shows a stop codon readthrough event.

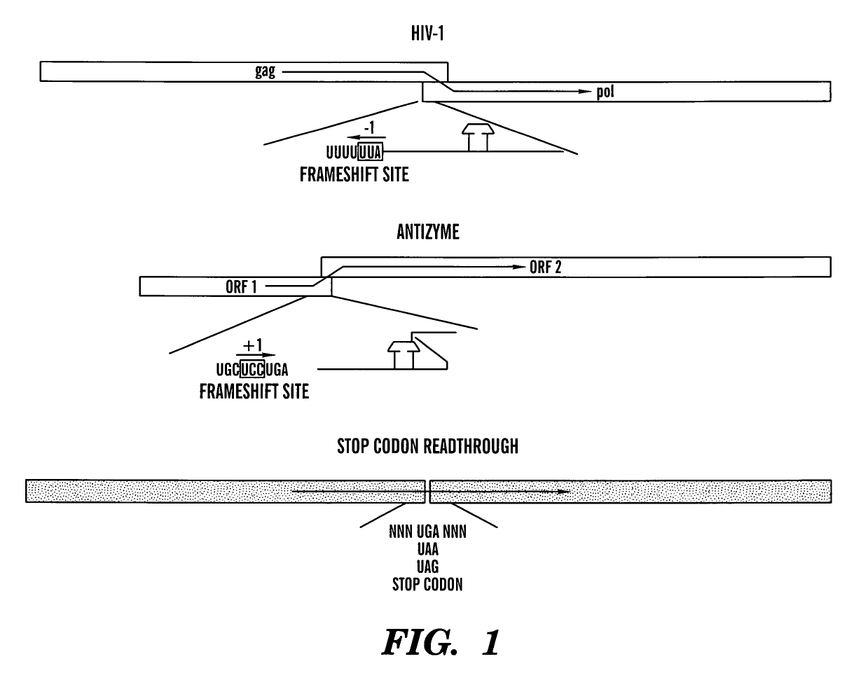

FIG. 2. Schematic representation of the various frameshifting constructs. The NP gene contains a unique SphI site between the NP₁₄₇₋₁₅₅ and NP₃₆₆₋₃₇₄ epitopes into which paired oligonucleotides were inserted representing various frameshifting elements in addition to the appropriate negative and positive control sequences. For many constructs a version of the NP gene was employed, immediately preceding NP₃₆₆₋₃₇₄ into which DNA encoding the Ova₂₅₇₋₂₆₄ epitope was inserted. All constructs were recombined into the vaccinia virus (VV) genome to allow expression in vitro and in vivo.

FIG. 3. The HIV frameshifting element directs expression and in vitro presentation of NP₃₆₆₋₃₇₄ that has been shifted to the −1 reading frame. Sequence containing the wild-type HIV frameshifting element was inserted into the SphI site of NP, shifting all downstream NP-encoding sequence, including NP₃₆₆₋₃₇₄, into the RF−1 (HIV-FS). Also inserted were positive control sequence, maintaining downstream sequence in RFO (HIV-IF) and negative control sequence designed to prevent the possibility of frameshifting (HIV-NC). The indicated target cell lines were infected with rVVs expressing these constructs as well as a negative control VV (VV-NC) and a second positive control (NP-expressing) VV (NP Vac) and then tested for epitope expression in a standard ⁵¹Cr-release assay, using NP₃₆₆₋₃₇₄-primed spleen cells, infra. Effector:target ratios (from left to right) for both cells were 80, 27, 9, and 3.

FIG. 4. The TK frameshifting element directs expression and in vitro presentation of Ova₂₅₇₋₂₆₄ that has been shifted to RF+1. The TK frameshifting element was inserted into the SphI site of NP/Ova₂₅₇₋₂₆₄, shifiting all downstream NP/Ova₂₅₇₋₂₆₄-encoding sequence, including Ova₂₅₇₋₂₆₄, into RF+1 (TK-FS). Negative and positive control TK sequences (TK-NC, and TK-IF respectively) were also inserted. L-Kb cells were infected Overnight with rVVs expressing these three constructs, along with a negative control VV (VV-NC). The infected cells were then fixed and tested for the ability to stimulate production of β-galactosidase by the Ova₂₅₇₋₂₆₄-specific B3Z hybridoma and the BWZ control cell line, infra. Note that the rVVs used for this and subsequent assays express β-glucuronidase as a marker for recombination, rather than β-galactosidase. Similar observations were made with three additional assays.

FIG. 5. The AZ frameshifitng element directs expression and in vitro presentation to the B3Z hybridoma of Ova₂₅₇₋₂₆₄ that has been shifted to RF+1 but a mutated version of the element does not. The AZ frameshifting element was inserted into the SphI site of NP/Ova₂₅₇₋₂₆₄, shifting all downstream NP/Ova₂₅₇₋₂₆₄-encoding sequence, including Ova₂₅₇₋₂₆₄, into RF+1 (AZ-FS). A negative (AZ-NC) control sequence was also inserted, as was a version of the frameshifting element in which the stimulating stop codon was mutated (AZ-Stop). rVVs expressing these constructs, as well as synthetic Ova₂₅₇₋₂₆₄ peptide were tested for the ability to stimulate the B3Z (Ova₂₅₇₋₂₆₄-specific) and BWZ (negative control) cell lines.

FIG. 6. AZ-IF and AZ-Stop direct sufficient expression of Ova₂₅₇₋₂₆₄ for presentation to Ova₂₅₇₋₂₆₄-specific spleen cells. The rVVs described in FIG. 4 and the AZ-IF positive control were tested for the ability to sensitize L-Kb target cells for killing by NP₅₀₋₅₇- and Ova₂₅₇₋₂₆₄-specific spleen cells, developed as described in Materials and Methods. Effector:target ratios are 39:1, 13:1 and 4.3:1 for the NP₅₀₋₅₇-specific assay (left panel) and 90:1, 30:1 and 10:1 for the Ova₂₅₇₋₂₆₄-specific assay (right panel).

FIG. 7. AZ-IF and AZ-Stop both prime mice for an Ova₂₅₇₋₂₆₄-specific response as measured by a standard ⁵¹Cr-release assay. C3FeB6F1/J (H-2^{k} and H-2^{b}) mice were injected i.p. with equivalent doses of the indicated rVVs. Spleen cells were then restimulated in vitro and then tested for the ability to lyse L-K^{b} target cells infected with rVVs expressing NP₅₀₋₅₇ (left panel) o a₂₅₇₋₂₆₄ (right panel), infra. Two separate experiments are shown. In Experiment 1, mice were immunized with 10⁷ pfu of each rVV and effector:target ratios are 100, 33, and 11 for the NP₅₀₋₅₇-specific assay (left panel) and 123, 41, and 14 for the Ova₂₅₇₋₂₆₄-specific assay. In Experiment 2, mice were immunized with 10⁶ pfu of each rVV and effector:target ratios are 50, 17, and 5.6 for the NP₅₀₋₅₇-specific assay (left panel) and 69, 23, and 7.6 for the Ova₂₅₇₋₂₆₄-specific assay.

FIG. 8. AZ-IF and AZ-Stop both prime mice for an Ova₂₅₇₋₂₆₄-specific response as detected by interferon-γ-based ELISPOT analysis. Mice were immunized as described in FIG. 7 and then spleen cells were subjected to standard ELISPOT analysis to assess the magnitude of the in vivo NP₅₀₋₅₇- and Ova₂₅₇₋₂₆₄-specific responses.

### DESCRIPTION OF THE INVENTION

CD8⁺ T cells (T_{CD8+}) respond to antigen in the form of short (8-10 amino acids) peptides (termed epitopes) bound to MHC class I molecules and constitute an important defense against intracellular pathogens by limiting spread following infection (Townsend and Bodmer, Annual Review of Immunology 7:601, 1989; Yewdell and Bennink, Advances in Immunology, 52:1, 1992; Germain and Margulies, Annual Review of Immunology 11:403, 1993; Palmer and Cresswell, Annu Rev Immunol, 16:323, 1998). These epitopes are generated through proteolysis and loaded onto MHC class I molecules within the cell. Once epitope/MHC class I complexes have been formed, they are transported to the cell surface where they can be contacted by T_{CD8+} bearing receptors of the correct specificity.

Since most cells express class I constitutively they are capable of activating a T_{CD8+} response if provided antigen. The percentage of T_{CD8+} capable of being triggered is very low in a naive animal (perhaps on the order of 0.001-0.1%), but upon stimulation this epitope-specific population expands rapidly and very large numbers. In extreme cases, the fraction of all T_{CD8+} that is specific for a single peptide antigen can be greater than 50% (Butz and Bevan, Immunity 8, 167-175, 1998; Murali-Krishna et al., Immunity 8, 177-187, 1998). Even much lower levels of expansion are easily measured with routine assays (Busch et al., Journal of Experimental Medicine 188,61-70, 1998; Busch et al., Immunity 8, 167-175, 1998; Flynn et al., Immunity 8, 683-91, 1998).

T_{CD8+} recognition is very specific, with slight changes in the peptide sequence usually leading to loss of recognition. Thus, individual T_{CD8+} generally respond to a single peptide sequence within a pathogen. This is certainly the case with the expression system of the present invention. The sensitivity of T_{CD8+} is remarkable with only tens to hundreds of copies of the same peptide required at the surface of a single cell for activation (Christinck, et al, Nature, 352:67, 1991; Schodin, et al, Immunity 5, no. 2:137, 1996; Bullock and Eisenlohr, Journal of Experimental Medicine, 184:1319, 1996). This number of peptides is derived from an amount of protein that is undetectable by standard biochemical methods (Bullock and Eisenlohr, Journal of Experimental Medicine, 184:1319, 1996; Wherry, et al, J Immunol 163, No. 7:3735, 1999). Indeed, it is now clear that a sufficient supply of peptide is derived from proteins that are not even the products of conventional gene expression. For example, T_{CD8+} have been shown to respond to “cryptic” epitopes encoded outside of conventional open reading frames (Coulie et al, Proceedings of the National Academy of Sciences USA, 92:7976, 1995; Guilloux, et al, Journal of Experimental Medicine 183: 1173, 1996; Uenaka, Journal of Experimental Medicine, 180:1599, 1994; Robbins, et al, J Immunol 159, no. 1:303, 1997) and within alternative reading frames (Mayrand and Green, Immunol Today 19, no. 12:551, 1998; Wang, et al, Journal of Experimental Medicine 183:1131, 1996), with expression demonstrated or suspected to be driven by cryptic promoter activity (Uenaka, Journal of Experimental Medicine 180:1599, 1994), alternative mRNA splicing ((Coulie et al, Proceedings of the National Academy of Sciences USA, 92:7976, 1995; Guilloux, et al, Journal of Experimental Medicine 183: 1173, 1996, Uenaka, Journal Experimental Medicine, 180:1599, 1994), initiation of translation at non-AUG codons (Malarkannan, S., et al., J. of Exper. Med. 182: 1739, 1995; Malarkannan, S., et al., Immunity 10, no. 6: 681, 1999), and initiation at internal AUG codons (Bullock and Eisenlohr, Journal of Experimental Medicine, 184:1319, 1996; Bullock, et al, Journal of Experimental Medicne, 186:1051, 1997) Such findings suggest that the general conception of foreign and self-antigens should be broadened to include these kinds of proteins. The extent to which aberrant gene expression drives immune responses is not known but, given the sensitivity of T_{CD8+}, the contribution could be considerable. Further, in cases when potential targets for the immune system may be limited, such as latently-infected or transformed cells, the contribution could be critical.

The present invention relates to an unconventional form of gene expression, ribosomal frameshifting, which, though suspected of being active in the generation of cryptic epitopes (Malarkanna, et al, Journal of Experimental Medicine, 182:1739, 1995; Malarkannan, et al Immunity10, no. 6:681), has not been rigorously investigated in this regard. Translational frameshifting occurs when the ribosome, in the course of translating an mRNA, does not follow the normal triplet rules for decoding and shifts into either the −1 or +1 reading frame. Subsequent triplet translation in the new frame yields a transframe protein with novel amino acid sequence encoded after the shift site, a potential source of epitopes encoded by non-standard reading frames. Although the reliability of triplet reading is generally high, such frameshift errors are detectable with certain sequences such as homopolymeric runs of nucleotides or slowly decoded codons especially prone to such errors (Gallant and Lindsley, Biochem Soc Trans, 21, no. 4:817, 19931 Weiss, et al, Progress in Nucleic Acids Research 39:159, 1990; Fox and Brummer, Nature, 288, no. 5786:60: Atkins, et al, Emb J 2, no. 8:1345, 1983). Errors in frame maintenance have typically been studied by using frameshift mutants (bases added or deleted from coding sequences) (Weiss, et al, Progress in Nucleic Acids Research, 39:159, 1990; Fox and B. Weiss-Brummer, Nature 288, no. 5786:60, 1980; Atkins, et al, Proceedings of the National Academy of Sciences USA 69:1192, 1972; Farabao , F. J., Prog Nucleic Acid Res Mol Biol 64:131, 2000; Horsburgh, et al, Cell, 86:949, 1996; Kurland, C. G., Academic Press. 97, 1979). The production of a small amount of full length product from such mutants results when a proportion of ribosomes spontaneously shift frame near the site of the mutation such that these ribosomes translate the rest of the coding sequence in the original reading frame. Although the methods to detect frameshift errors have relied on analysis of frameshift mutations, low level frameshifting errors also occur in decoding wildtype sequences.

A recent example of one high frequency translational frameshift error was discovered in the course of studying a frameshift mutation within the Herpes thymidine kinase (TK) gene (Hwang, et al, Proc. Natl Acad Sci USA, 91, no. 12:5461, 1994). Acyclovir resistant viral mutants have been isolated that contain an extra G added to a run of seven Gs within the thymidine kinase gene. Most ribosomes upon encountering this frameshift mutation continue triplet translation into the new frame and terminate at a nearby stop codon. However, approximately 1% of the ribosomes shift within the run of Gs, restoring the original reading frame and continue translation to produce full length protein (Horsburgh, et al, Cell, 86:949, 1996). One percent of the normal level of thymidine kinase protein is below the threshold for acyclovir sensitivity but is enough to reactivate latent virus. Consequently, this frameshift error allows the resistant virus to survive and avoid anti-viral therapy. Subsequent characterization of the error prone frameshift site revealed that the wildtype sequence of seven Gs also stimulated frameshifting to the same degree (Horsburgh, et al, Cell, 86:949, 1996). Thus even in the wildtype virus, approximately 1% of translating ribosomes likely shift into the +1 frame and terminate at a nearby stop codon generating a low level of aberrant TK protein product.

Another potential source of transframe protein (epitope) expression comes from programmed translational frameshifting. In contrast to errors in translational frame maintenance, programmed frameshifting occurs at particular sites and is utilized by the cell for gene expression (Atkins, et al, editors Cold Spring Harbor Press, NY. 67, 1 ; Farabaugh, P. J., Microbiol Rev 60, no. 1:103, 1996; Gesteland and Atkins, Annual Reviews in biochemistry 65:741, 1996). Such programmed frameshifting occurs at much greater levels than error prone frameshifting due to specific stimulatory cis acting sequences located within the mRNA. Stimulatory sequences, although quite variable, typically encompass the frameshift site, where ribosome and tRNAs shift relative to the mRNA, and often include adjacent sequences such as a downstream RNA stem loop or pseudoknot. A classic example is the Human Immunodeficiency Virus (HIV) which has overlapping gag and pol genes such that a −1 frameshift at a U-rich shift site (followed by a stem loop RNA structure) near the end of the gag gene is required for expression of the Gag-Pol fusion protein (Jacks, et al, Nature, 331, no. 6153;280, 1988). The frequency of translational frameshifting determines the ratio of Gag to Pol during infection, as this transframe product is the sole source of reverse transcritpase.

Whereas, programmed −1 frameshifting appears to be quite common in mammalian viruses, bacterial insertion sequences, and a few other classes of genes, few examples of programmed frameshifting are known to occur in cellular genes. The only known mammalian example occurs during translation of the ornithine decarboxylase antizyme (AZ) genes (Ivanov, et al, Genomics, 52, no. 2:119, 1998; Ivanov,, et al, Proc Natl Acad Sci USA 97, no. 9:4808, 2000; Matsufuji, et al, Cell, 80:51, 1995; Rom and Kahana, Proc Natl Acad Sci USA 91, no. 9:3959, 1994; Zhu, et al., J Biol Chem 274, no. 37:26425, 1999). AZ genes contain two overlapping open reading frames (ORFs) with the second downstream ORF in the +1 reading frame relative to the upstream ORF. The +1 translational frameshift required to produce full length antizyme is a sensor of polyamine levels. As antizyme is a potent inhibitor of ornithine decarboxylase (ODC, which carries out the rate limiting step in polyamine biosythesis), and also inhibits polyamine transport into the cell, polyamine stimulated frameshifting creates an autoregulatory loop to maintain appropriate intracellular concentrations of polyamines (Hay i, et al, Trends in Biochemical Science 21:27, 1996).

Programmed and error prone frameshifting have particularly high potential for expression of cryptic epitopes since, unlike other mechanisms that have thus far been investigated, it occurs at any point within the open reading frame. Frameshift sites, derived from the three different frameshifting cases described above, AZ (+1), HIV (−1) and the Herpes TK (+1), ranging in efficiency from 40% to less than 1%, were tested for their ability to induce immunologically detectable expression of two different T_{CD8+} epitopes. The results indicate that even extremely weak frameshifting elements can elicit T_{CD8+} responses in vitro and in vivo.

The present invention is a system for measuring recoding in vivo. This is due to the exquisite sensitivity and specificity with which T_{CD8+} recognize particular peptide sequences. If a particular sequence is placed in an alternative reading frame or beyond a stop codon, the activation of a T_{CD8+} specific for that sequence is a clear indication that the alternative reading frame has been translated or that the stop codon has been bypassed, even if either is a rare event. Critically, the present invention allows for the T_{CD8+} responses to be graded so that one is able to determine whether the level of recoding has been altered by introduction of a test compound.

Materials and Methods

Mice, Cell Lines and Chemicals. 6-to 8-week-old female C3H (H-2^{k}), C57BI/6 (H-2^{b}) and C3FeB6F1/J (H-2k and H-2^{b}) mice were purchased from Taconic Laboratories (Albany, N.Y.) or The Jackson Laboratory (Bar Harbor, Me.), and maintained in the Thomas Jefferson University Animal Facilities (Philadelphia, Pa.). The murine L929 (H-2^{k}; American Type Culture Collection (ATCC), Manassas, Va.) cells, L929 transfected with the K^{b} gene (L-K^{b} cells, kindly provided by Dr. Y. Paterson, University of Pennsylvania, Philadelphia), L929 transfected with the D^{b} gene (L-D^{b} cells, kindly provided by Drs. J. W. Yewdell and J. R. Bennink, National Institutes of Health, Bethesda, Md.), K-145 cells (Kindly provided by Dr. S. S. Teveia, Pennsylvania State University, Hershey, Pa.) and 143. (TK⁻) cells (CRL-8303; ATCC) for vac expansion and titration were maintained in DMEM (Cellgro Products, Fisher Scientific) supplemented with 5% FCS at 9% CO₂. EL-4.G7-OVA (a kind gift of Drs. J. W. Yewdell and J. R. Bennink), and EL-4 cells (kindly provided by Dr. E. C. Lattime, Cancer Institute of New Jersey, New Brunswick, N.J.) were maintained in RPMI 1640 (Cellgro) supplemented with 10% FCS, 10 μg/ml gentamicin, and 5×10⁻⁵ M 2-ME at 6% CO₂. The OVA₂₅₇₋₂₆₄/K^{b}-specific, LacZ-transfected T cell hybridoma, B3Z, and the fusion partner, BWZ.36 (kindly provided by Dr. Nilabh Shastri, University of California, Berkeley, Calif.) were maintained in RPMI 1640 supplemented with 10% FCS, 10 μg/ml gentamicin, and 5×10⁻⁵ M 2-ME (assay medium). All chemicals were purchased from Sigma (St. Louis, Mo.) unless otherwise noted.

Molecular constructs. Construction of the NP/Ova₂₅₇₋₂₆₄ gene has been described elsewhere (Wherry, et al, J Immunol 163, no. 7:3735, 1999). For the HIV, TK and AZ constructs, complimentary oligonucleotides (sequence of the sense strand shown below) were synthesized on an Applied Biosystems model 380C synthesizer such that when annealed they would have SphI compatible ends. They were ligated into SphI digested NP (HIV constructs) or NP/Ova₂₅₇₋₂₆₄ (TK and AZ constructs), both contained within modified versions of the pSC11 plasmid (Chakrabarti, et al, Molecular Cellular Biology,5:3403, 1985) used for homologous recombination into the VV genome. After transformation into E. coli strain SU1675, DNA sequences were verified by autothermocycler sequencing, and plasmids were purified using the Qiagen Midiprep Kit (Valencia, Calif.) according to manufacturer's specifications. The synthetic oligonucleotides used are as follows: HIV Frameshift (HIV-FS): 5′ C GCT AAT TTT TTA GGG AAG ATC TGG CCT TCC TAC AAG GGA AGG CCA GGG AAT TTT CTT CAT G 3′ (SEQ. ID. NO: 1); HIV Negative Control (HIV-NC): 5′ C GCT AAT TTT CTA GGG AAG ATC TGG CCT TCC TAC AAG GGA AGG CCA GGG AAT TTT CTT CAT G 3′ (SEQ. ID. NO: 2); HIV In-Frame (HIV-IF): 5′ C GCT AAT TTT TTA GGG AAG ATC TGG CCT TCC TAC AAG GGA AGG CCA GGG AAT TTT CTT CCA TG 3′ (SEQ. ID. NO: 3); TK Frameshift (TK-FS): 5′ C CTG GCT CCT CAT ATC GGG GGG GGA GGC TGG GAG CTC AGC ATG 3′ (SEQ. ID. NO: 4); TK Negative Control (TK-NC): 5′ C CTG GCT CCT CAT ATC GGA GGC TGG GAG CTC AGC ATG 3′ (SEQ. ID. NO: 5); TK In-Frame (TK-IF): 5′ C CTG GCT CCT CAT ATC GGG GGG GAG GCT GGG AGC TCA GCA TG 3′ (SEQ. ID. NO: 6); AZ Frameshift (AZ-FS): 5′ C TGG TGC TCC TGA TGT CCC TCA CCC ACC CCT GAA GAT CCC AGG TGG GCG AGG GAA CAG TCA GCG GGA TCA CAG CGC ATG 3′ (SEQ. ID. NO: 7); AZ Stop Frameshift (AZ-STOP): 5′ C TGG TGC TCC GGA TGT CCC TCA CCC ACC CCT GAA GAT CCC AGG TGG GAG AGG GAA CAG TCA GCG GGA TCA CAG CGC ATG 3′ (SEQ. ID. NO: 8); AZ Negative Control (AZ-NC): 5′ C TGG TGC TCC TGA TGT CCC TCA CCC ACC CCT GAA GAT CCC AGG TGG GCG AGG GAA CAG TCA GCG GGA TCA CAG CCG CAT G 3′ (SEQ. ID. NO: 9); AZ In-Frame (AZ-IF): 5′ C TGG TGC TCC GGA TGT CCC TCA CCC ACC CCT GAA GAT CCC AGG TGG GCG AGG GAA CAG TCA GCG GGA TCA CAG GCA TG 3′ (SEQ. ID. NO: 10). In addition, the TK and AZ constructs were excised from pSC11 via Sal I/Not I cutting and cloned into pSC11 containing β-glucuronidase instead of β-galactosidase in order to allow use of the β-galactosidase-producing T hybridoma B3Z (below). The plasmids were recombined into the vaccinia virus genome and confirmed by sequencing as described elsewhere (Yellen-Shaw, et al, Journal of Immunology, 158:1727, 1997). All enzymes were purchased from New England Biolabs (Beverly, Mass.).

Viruses. The recombinant vaccinia viruses encoding NP_{(M)50-57} and NP_{(M)366-374} have been previously described (Wherry, et al, J Immunol 163, no. 7:3735, 1999). The OVA_{(M)257-264} VV was a kind gift of Drs. Yewdell and Bennink. Recombinant viruses were made as described elsewhere (Eisenlohr, et al, Journal of Experimental Medicine 175:481, 1992). Expression of all the NP-based constructs was driven by the vaccinia P₇₅ (early/late) promoter. Plasmids were introduced into the vaccinia genome by homologous recombination in CV-1 cells and triple plaque purified in 143B cells in the presence of 5 mg/ml 5-bromo-2-deoxyuridine (Boehringer Mannheim, Indianapolis, Ind.) and then expanded and titered on 143B HuTK-cells.

CTL generation. NP₅₀₋₅₇-, NP₃₆₆₋₃₇₄-or OVA₂₅₇₋₂₆₄ specific CTL populations were generated by immunization of C3H, C57B1/6 and/or C3FeB6Fl/J mice as previously described (Eisenlohr, et al, Journal of Experimental Medicine 175:481, 1992; Yellen-Shaw, et al, Journal of Immunology 158:3227, 1997). Briefly, mice were immunized i.p. with 10⁶ or 10⁷ pfu of NP_{(M)50-57}, NP_{(M)366-374} or OVA_{(M)257-264} rVV virus in 400 ·l balanced salt solution with 0.1% BSA (BSS/BSA). Two weeks later, spleens were harvested, homogenized and restimulated with A/PR/8/34 influenza virus to expand the NP₅₀₋₅₇- and NP₃₆₆₋₃₇₄-specific population or irradiated (10,000 cGy) EL-4.G7-OVA cells to expand the Ova₂₅₇₋₂₆₄-specific population. Recombinant IL-2 (20 U/ml, AIDS Research and Reference Reagent Program, National Institutes of Health) was included in the Ova₂₅₇₋₂₆₄ restimulation culture.

⁵¹Cr-release assays. ⁵¹Cr-release assays were carried out as previously described (Wherry, et al, J. Immunol 163, no. 7:3735, 1999; (Eisenlohr, et al, Journal of Experimental Medicine 175:481, 1992; Yellen-Shaw, et al, Journal of Immunology 158:3227, 1997). Briefly, target cells (K-145 and L-D^{b} for the HIV constructs) and L-K^{b} (for the AZ constructs) were infected at 10 plaque-forming units of virus/cell. Four hours later, the cells were pelleted and pulsed with 100 μCi/10⁶ cells of Na₂⁵¹CrO₄ (Amersham Pharmacia Biotech, Piscataway, N.J.) in 50 μl of the appropriate growth medium. Cells were washed 3 times with PBS, suspended in medium and combined with CTL at various ratios. After 4 h of co-incubation at 37° C., 100 μl were harvested from each well and percent specific ⁵¹Cr-release was determined by analysis in a gamma counter (Pharmacia, Sweden).

β-galactosidase-based T hybridoma stimulation assays. Assays for epitope expression based upon use of the B3Z T hybridoma that produces β-galactosidase upon activation, have been described previously (Wherry, et al, J. Immunol 163, no. 7:3735, 1999). Briefly, 5×10⁴ L-K^{b} cells were infected in six separate wells with the appropriate rVVs at 10 pfu/cell or pulsed with synthetic Ova₂₅₇₋₂₆₄ peptide (10⁻⁹ M). After one hour, wells were washed with PBS and overlayed with B3Z (Ova₂₅₇₋₂₆₄-specific) or BWZ.36 cells at 5×10⁴/well. After overnight incubation, β-galactosidase production was assessed using the fluorogenic substrate methyl umbelliferone-βgalactoside as described by Sanderson and Shastri (Sanderson and N. Shastri, Internal Immunology 158:3227, 1997).

In vivo priming assays. ⁵¹Cr-release-based, priming assays were carried out as described previously (8). C3FeB6F1/J were infected i.p. with 10⁶ or 10⁷ pfu of various rVVs in 400 μl BSS/BSA. Spleens were removed after 14 days and restimulated essentially as described above, except that spleen populations were adjusted to the same cell density for restimulation in a given experiment. For the assay these, populations were tested for the ability to lyse NP_{(M)50-57} - or NP_{(M)Ova257-264}-infected L-K^{b} cells as described above.

ELISPOT assays. The ELISPOT assays were performed essentially as described (Wherry, et al, J. Immunol 163, no. 7:3735, 1999) with slight modifications. Mice were immunized as described above. After 14 days, spleen cells were homogenized, red cells were lysed, and plated at various densities in 96-well ELISPOT plates coated 1 day previously with 20 μg/ml of monoclonal anti-interferon-γ (HB170, ATCC). Wells then received irradiated (10,000 cGy) L-K^{b} cells, IL-2 at 40 U/ml, 0.165 μg/ml β₂-microblobulin (Scripps Institute, La Jolla, Calif.) and nothing, synthetic NP₅₀₋₅₇ peptide (10⁻⁹ M) or synthetic Ova₂₅₇₋₂₆₄ (10⁻⁸) M. Plates were incubated 18 hr at 37° C., 6% CO₂ and then washed extensively (9 times) with PBS+0.25% Tween-20. Wells were then incubated with biotinylated anti-interferon-γ (BD PharMingen, San Diego, Calif.) at 4 μg/ml for 2 hours at rt. After extensive washing (6 times), 10 μg/ml HRP-avidin D (Vector Laboratories, Burlingame, Calif.) was added to each well and incubated 2 h at rt. After 5 washes with PBS+0.25% Tween-20 and one wash with water, spots were developed using 3.3′ diaminobenzidine and β-chloronaphthol dissolved in methanol and added to 10 ml of PBS containing 20 μl H₂O₂ (30%). Spots were counted using a dissecting microscope.

Results

The Base Construct and its Derivatives

The base construct described herein represents an example of a construct that is used in determining the efficacy of a recoding event. The scope of the invention is not limited to this example, the example is used to illustrate the technology of the present invention, which is a more sensitive method of detection of a recoding event. Those skilled in the art are familiar with recombinant techniques so that any reporter gene that contains a sequence(s) known to elicit a CD8⁺ T-cell response can be engineered into an expression vector for the purposes of testing a recoding event.

A sequence that is suspected of causing recoding is inserted into the SphI site in the gene construct, this insertion is composed so that recoding must take place in order for the two downstream MHC I restricted epitope sequences to be expressed. For example, a portion of the antizyme gene is inserted into the SphI site. This insertion now places a portion of the gene downstream of the insertion in the +1 reading frame. In order for these two epitopes to be expressed, the translating ribosome must shift into the +1 reading frame. The presentation of upstream epitopes is unaffected by the insertion and serves as a positive control for expression. Data from cell free translation assays have shown that this antizyme sequence will induce a significant level of frameshifting (Grentzmann et al., Rna 4, 479-86, 1998). The T-cell based assays of the present invention confirm the results obtained in the cell free translation assays, both in vitro and in vivo.

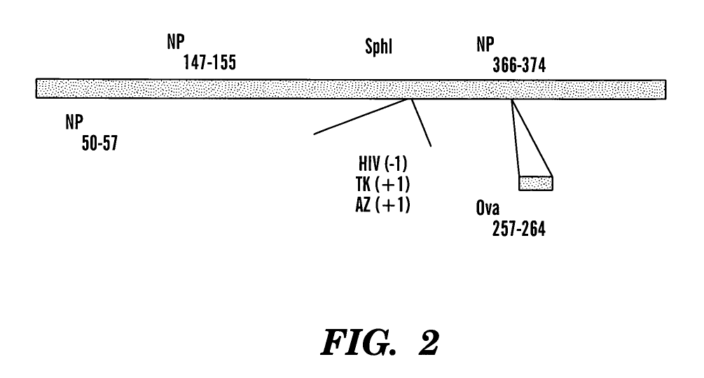

At the core of each construct is the open reading frame of the A/PR/8/34 influenza virus nucleoprotein (NP) (FIG. 2). This protein was selected because it contains three well-defined MHC class I-restricted itopes, NP₅₀₋₅₇ (H-2K^{k}-restricted), NP₁₄₇₋₁₅₅ (H-2K^{d}-restricted), and NP₃₆₆₋₃₇₄ (H-2-D^{b}-restricted). Further, no evidence for internal ribosomal entry sites were found within NP, which, as described for translation of poliovirus mRNA (McBratney, et al, Current Opinion in Cell Biology, 5:961, 1993), cause the ribosome to engage message at an interior site (termed a “landing pad”) rather than the 5′ cap, and would confound interpretation of results if positioned beyond a putative frameshift element. Control constructs, infra, confirmed the validity of the NP gene in this respect. Established frameshifting elements from the HIV gag-pol interface, the herpes simplex virus thymidine kinase gene (TK), and the mammalian antizyme gene (AZ), as well as respective control sequences, were placed at a unique SphI site, downstream of the NP₅₀₋₅₇ and NP₁₄₇₋₁₅₅ epitopes and upstream of the NP₃₆₆₋₃₇₄. This location is sufficiently downstream from the 5′ terminus of the message to eliminate translational reinitiation following termination as a potential complication, since reinitiation appears to be a viable mechanism only during the early phases of translation, thought to be due to the gradual loss of initiation factors during elongation of the translation product (Kozak, M. Molecular and Cellular Biology, 7:3438, 1987; Luukkonen, et al, Journal of Virology, 69:4086, 1995).

For two of the frameshifting elements (TK and AZ) NP was modified by inserting the sequence to encode the Ova₂₅₇₋₂₆₄ epitope (H-2K^{b}-restricted) adjacent to the NP₃₆₆₋₃₇₄ as depicted (FIG. 2). This was done because responses to the Ova₂₅₇₋₂₆₄ epitope are somewhat more reliable than those to NP₃₆₆₋₃₇₄, and also because of the existence of useful and sensitive reagents specific for the Kb/Ova₂₅₇₋₂₆₄ complex. Inserted elements were positioned in such a way that a −1 frameshifting event, in the case of the HIV element, or a +1 frameshifting event, in the cases of the TK and AZ elements, would be required for continued translation of NP in the downstream open reading frame. These constructs were then recombined into the vaccinia virus (VV) genome and the series of recombinant VVs (rVVs) tested in in vitro and in vivo assays.

The HIV meshifting Element

The frameshift stimulatory sequences excerpted from the gag-pol frameshift window of the HIV genome (Jacks, et al, Nature 331, no. 6153:280, 1988) were first tested. Retroviral frameshifting occurs at heptanucleotide slippery sequence motif of the form X XXY YYZ (where XXX is a repeat of any nucleotide, Y is U or A, and Z is U, A, or C) followed by a secondary structure of either a simple stem loop in the case of HIV (Parkin, et al, J Viro 66, no. 8:5147, 1992) or a pseudoknot as in Mouse Mammary Tumor Virus (Chamirrim et al, Proceedings of he Natinal Academy of Scienses USA 89-713, 1992; Gonzalez and Tinoco, J Mol Biol 289, no. 5:1267, 1999; Hizi, et al, Proc Natl Acad Sci USA 84, no. 20:7041, 1987). In these examples, the two tRNAs in the A and P sites of the ribosome slip in tandem one base with respect to the mRNA and re-basepair to mRNA at an overlapping matched codon to continue translation in the new reading frame.

The HIV frameshift element, U UUU UUA followed by a stem loop, has been studied extensively and shown to direct approximately 5% of the translating ribosomes to shift into the −1 reading frame (different methods for measuring frameshifting reveal different frameshift frequencies with results varying between 0.7 and 12%, although most studies suggest frameshifting around 5%). (Parkin, et al, J Viro 66, no. 8:5147, 1992; Cassan, et al, J Virol 68, no. 3:1501, 1994; Reil, et al, J Virol 67, no. 9:5579, 1993; Vickers and D. J. Ecker, Nucleic Acids Res 20, no. 15:3945, 1992). This element was placed into the SphI site of the NP gene (see FIG. 2), shifting the downstream NP sequence in the −1 frame (RF−1) to create the HIV-FS construct. To provide a negative control (HIV-NC), the slippery site was mutated to prevent tRNA repairing in the −1 frame while the positive control sequence (HIV-IF) maintains downstream NP sequence in the standard reading frame (RFO).

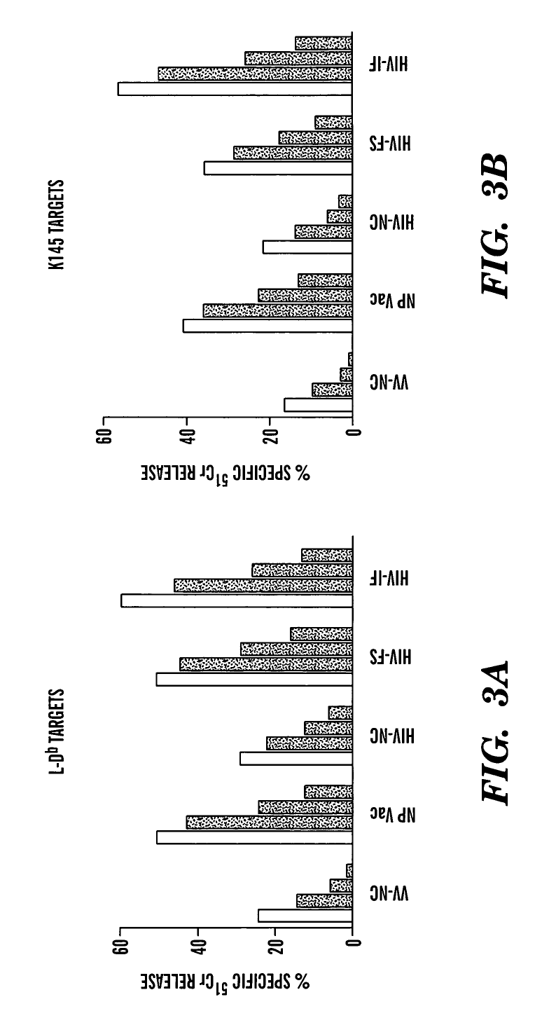

rVVs expressing these constructs were then tested for the ability to sensitize NP₃₆₆₋₃₇₄-specific T_{CD8+} in a conventional ⁵¹Cr-release assay. Two different cell lines expressing the appropriate MHC class I molecule (H-2D^{b}) were infected with equal doses of the various rVVs. After loading with ⁵¹Cr, the target cells were combined with NP₃₆₆₋₃₇₄-specific T_{CD8+} that were prepared as described supra and, four hours later, supernatants were harvested to assess cell lysis. FIG. 3 shows that the mutant negative control sequence (HIV-NC) sensitizes target cells for killing only slightly better than a control virus (VV-NC) that does not contain sequence encoding the NP₃₆₆₋₃₇₄ epitope. The slight activation observed with the HIV-NC reflects residual frameshift activity from the altered frameshift window. In contrast, the wild-type frameshifting element (HIV-FS) permits target cell lysis at levels comparable to the positive controls (NP Vac and HIV-IF). This general pattern was observed for both cell types. Thus, T_{CD8+} are capable of recognizing an epitope that is expressed only if ribosomal frameshifting occurs.

The TK Frameshifting Element

One naturally occurring error prone frameshift site that has been identified is within the mutated thymidine kinase (TK) gene of an acyclovir-resistant human herpes simplex virus (Horsburgh, et al, Cell 86:949, 1996; Hwang, et al, Proc Natl Acad Sci USA 91, no. 12:5461, 1994). This sequence (8 consecutive guanosine residues) in the absence of other frameshift stimulators, such as a pseudoknot, induces a level of +1 frameshifting of approximately 1%. Of note, the wild type sequence (7 consecutive guanosine residues) is a comparably active slippery site (Horsburgh, et al, Cell 86:949, 1996). Thus, this frameshift element also likely operates during translation of wild-type TK, creating a low level of aberrant protein and reducing slightly the yield of wild-type protein. Such natural, “unintentional” frameshifting elements are obviously of particular interest with respect to the expression of cryptic T_{CD8+} epitopes.

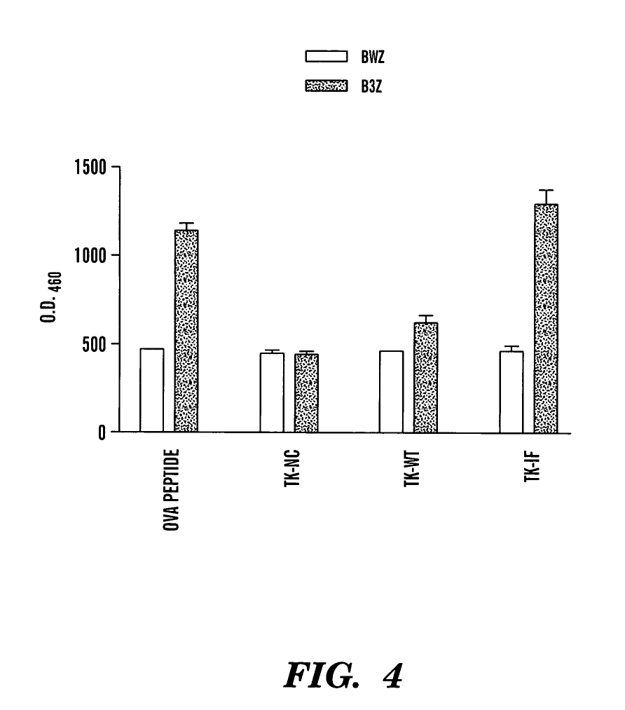

The frameshifting element derived from the mutant (TK-FS), as well as control sequences, in which the G run required for frameshifting was deleted (TK-NC), were tested following insertion into the SphI site of the NP/Ova₂₅₇₋₂₆₄ construct (see FIG. 2) and recognition of Ova₂₅₇₋₂₆₄ was monitored. In this case, a Kb/Ova₂₅₇₋₂₆₇- specific T cell hybridoma was employed that produces β-galactosidase upon activation rather than mouse-derived T_{CD8+}, eliminating the need for ⁵¹Cr-loading of the target cells. Target cells were infected with the rVVs indicated in FIG. 4, and co-incubated overnight with either the Kb/Ova₂₅₇₋₂₆₄-specific hybridoma B3Z, or a negative control cell line, BWZ, which has the potential to produce β-galactosidase but lacks the appropriate specificity. As can be seen, levels of β-galactosidase production by the two hybridomas were comparable when TK-NC was utilized, while there was a clear difference between β-galactosidase production with the positive control (TK-IF). As expected, the TK frameshifting element (TK-FS) is associated with a low but significant level of Kb/Ova₂₅₇₋₂₆₄-specific recognition, a finding that was observed with three additional assays. As described infra, this is a level of epitope expression that is clearly influential in vivo.

The AZ Frameshifting Element

The final frameshifting element studied was derived from the mammalian antizyme (AZ) gene. Under conditions of cell-free translation, the AZ element directs +1 frameshifting with an efficiency of 3-18% (33) and between 20 and 40% in tissue culture cells, with the level of frameshifting being controlled by polyamine concentration (Grentzmann, et al, Rna 4, no. 4:479, 1998). This high level frameshifting is stimulated by an adjacent stop codon in the 0 frame, as well as, RNA sequences 5′ and an RNA pseudoknot 3′ of the shift site (Ivanov, et al. Genomics 52, no. 2:119, 1998; Matsufuji, et al, Cell, 80:51, 1995). Several variations of the antizyme frameshift element designed to reveal differing levels of frameshifting were cloned upstream from the Ova₂₅₇₋₂₆₄ epitope.

First, the AZ frameshifting element (AZ-FS) lacking the upstream stimulatory element (causing about a two fold reduction in frameshifting from the wildtype) was cloned upstream of the Ova₂₅₇₋₂₆₄ epitope such that a +1 translational frameshift is required for expression. Second, the same construct was created with the stop codon mutated (AZ-Stop) reducing frameshifting to less than 1% (Matsufuji, et al, Cell, 80:51, 1995). Finally, an in frame positive control (AZ-IF) with the stop codon mutated to allow for full expression of the Ova₂₅₇₋₂₆₄ epitope, and a negative control (AZ-NC) with the Ca₂₅₇₋₂₆₄ epitope in the −1 fra to eliminate expression was constructed and frameshifting levels assessed in vitro and in vivo.

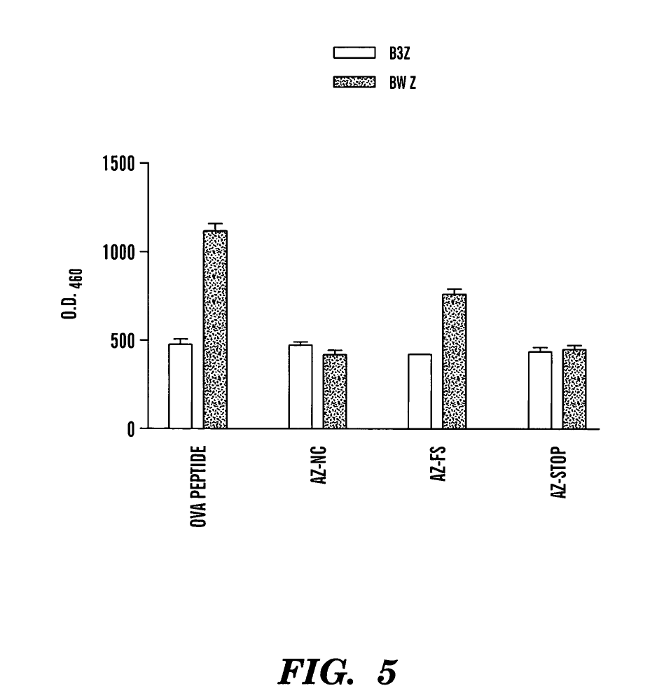

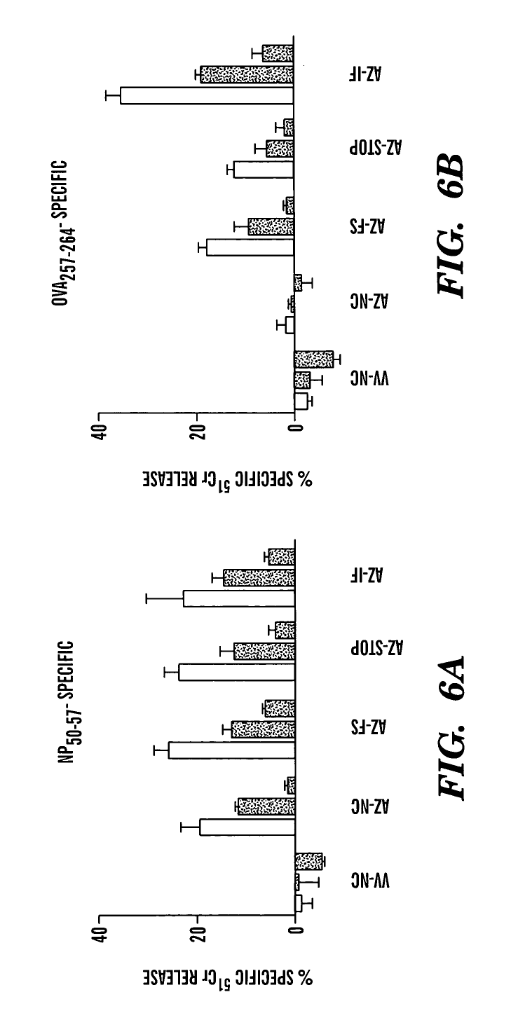

For in vitro assays, the β-galactosidase-producing T hybridoma system was first employed. As can be seen in FIG. 5, there was a strong specific response to a synthetic version of the Ova₂₅₇₋₂₆₄ epitope and to the 5′ deleted frameshift construct (AZ-FS), but, in many attempts, specific recognition of the mutant construct (AZ-Stop) was not detected. However, when a standard ⁵¹Cr-release assay was performed as described for the HIV element, in addition to AZ-IF and AZ-FS, the AZ-Stop construct was consistently recognized, as demonstrated in FIG. 6. NP₅₀₋₅₇-specific T_{CD8+} was also employed in the assay, which confirmed equivalent infection of the target cells. This control becomes much more important in assessing in vivo activation (infra). The results in FIGS. 4-6 indicate that, under the conditions employed, the AZ-Stop element is an even weaker inducer of frameshifting than the TK-FS element and yet will still elicit a detectable immune response in vitro, depending upon the assay employed.

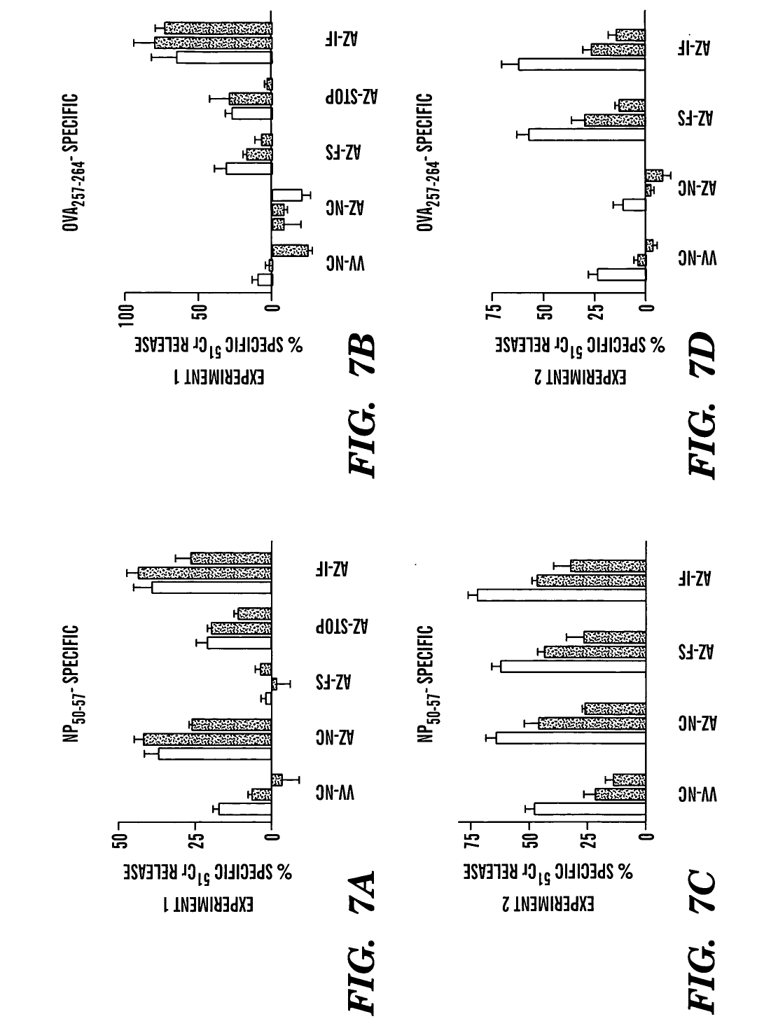

To test whether AZ frameshifting constructs would be sufficiently active in vivo, mice were immunized with equivalent infectious doses of the same rVVs. After 2 weeks, spleen cells were removed, restimulated in vitro and then tested in a standard ⁵¹Cr-release assay for the ability to recognize epitope-expressing target cells. NP₅₀₋₅₇-specific killing was measured in order to assess the level of priming that was achieved with each test construct. Equivalent priming by all rVVs is often difficult to achieve for reasons not fully understand. Thus, two such experiments are shown in FIG. 7.

Experiment 1 demonstrates that, despite a slightly lower level of priming for an NP₅₀₋₅₇-specific response compared to the positive controls, AZ-Stop clearly elicits an Ova₂₅₇₋₂₆₄-specific response. Similar results were observed in three additional assays where this virus was included and sufficient priming was observed for all of the key constructs. Also shown in Experiment 1 is the clear priming by AZ-FS for an Ova₂₅₇₋₂₆₄-specific response, despite undetectable priming for an NP₅₀₋₅₇-specific response. With stronger priming by this construct (as assessed by NP₅₀₋₅₇-specific killing), Ova₂₅₇₋₂₆₄-specific killing would be much higher, reflective of high level frameshifting. This prediction is borne out by the results of Experiment 2, where all of the AZ constructs primed for an equivalent NP₅₀₋₅₇ response and Ova₂₅₇₋₂₆₄-specific killing by cells from AZ-FS mice was as high as that by cells from AZ-IF-immunized mice. Similar results were obtained in three additional assays where NP₅₀₋₅₇-specific priming was high for both AZ-FS and AZ-IF.

In order to attain a more quantifiable result from in vivo priming, a standard interferon-γ-based ELISPOT assay was used to measure the level of T_{CD8+} expansion in vivo. In this case, spleen cells from primed mice were removed and restimulated with peptide pulsed cells, and the number of interferon-γ-producing (epitope-specific) cells assessed as described supra. As with the ⁵¹Cr-release assay, both NP₅₀₋₅₇- and Ova₂₅₇₋₂₆₄-specific responses were monitored.

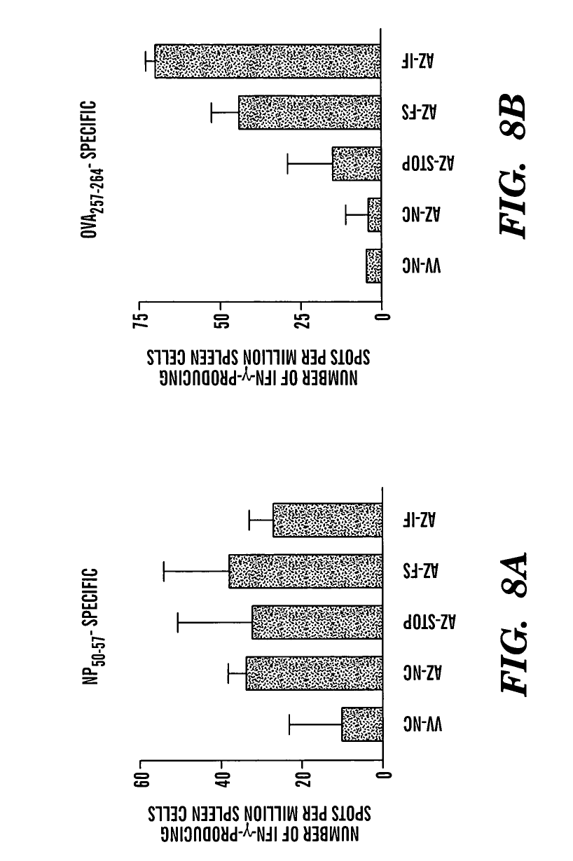

FIG. 8 shows results predicted by those of FIG. 7. Priming for the NP₅₀₋₅₇ response by all of the AZ constructs was equivalent, while responses to Ova₂₅₇₋₂₆₄ varied depending upon the construct being tested. Again, Ova257-264 responses to AZ-IF, AZ-FS, and AZ-Stop were observed. Thus, frameshifting as measured by T_{CD8+} activation is quite active in vivo, and even a very low level frameshifting that elicits marginal T cell activation in in vitro assays, elicits significant T_{CD8+} proliferation in vivo.

Efficacy of a Test Compound

When analyzing a test compound for the efficacy of recoding in vivo, the test compound is administered to the mice before (the length of time before is to be determined by the skilled artisan) or at the same time as the recombinant vector which contains the reporter gene. The timing of addition of the test compound is dependent on the particular properties of that compound, such as the rate of delivery to the relevant anatomical site, rate of transport across the cell membrane, the half-life, etc. In the example supra the vector is vaccinia virus and the reporter gene encodes influenza nucleotprotein epitopes. The dosage of the test compound, as well as the route of administration, are determined by those sed in the art at the time of analysis. Methods of administration most commonly used include, but are not limited to, intradermal, intramuscular, intraperitoneal, intravenous, subcutaneous, intranasal and orally. Administration of the test compound is systemic or local.

The toxicity of the test compound is also assessed in the in vivo system of the present invention. This is unique to the present invention in that the current methods available for testing a recoding event are limited to in vitro systems, such as tissue culture. By comparing the in vivo recoding event in the presence and absence of the test compound, the efficacy of recoding is determined.

When analyzing a test compound for the efficacy of recoding in vitro, the test compound is added to the cells expressing the appropriate MHC class I molecules before (the length of time before is to be determined by the skilled artisan) or at the same time as the recombinant vector which contains the reporter gene. The timing of addition of the test compound is dependent on the particular properties of that compound, such as rate of transport across the cell membrane, the half-life, etc. In the example supra the vector is vaccinia virus and the reporter gene encodes influenza nucleotprotein epitopes. A concentration range of the test compound is tested so that the most efficacious concentration is determined. The range at which a particular compound is tested will be determined by those skilled in the art at the time of testing. By comparing the recoding event in the presence and absence of the test compound, the efficacy of recoding is determined. Assays for in vitro analysis of efficacy are decided by those skilled in the art (for example the Shastri hybridoma assay wherein no radioactivity is required).

Discussion

Numerous reports have demonstrated the recognition of MHC class I-restricted epitopes that are not predicted to be expressed according to conventional mechanisms of gene expression. Such cryptic epitopes have been observed in a variety of tumors and viral infections (Mayrand, S. M. and W. R. Green, Immunol Today 19, no. 12:551, 1998) and a number underlying mechanisms have been identified or strongly implicated. These include cryptic promoter activity (Uenaka, et al, Journal of Experimental Medicine, 180:1599, 1994), aberrant mRNA splicing (Coulie, et al, Proceedings of the National Academy of Sciences USA, 92:7976, 1995; Guilloux, et al, Journal of Experimental Medicine 183:1173, 1996; Uenaka, et al, Journal of Experimental Medicine, 180:1599, 1994), translation initiation at a non-AUG codon (Malarkannan, et al, Journal of Experimental Medicine 182:1739, 1995; Malarkannan, et al, Immunity 10, no. 6:681, 1999), and translation initiation at an internal AUG (Bullock and Eisenlohr, Journal of Experimental Medicine 184:1319, 1996; Bullock, et al, Journal of Experimental Medicine 186:1051, 1997). To this list is added ribosomal frameshifting, a phenomenon in which the translating ribosome is directed into one of two alternative reading frames either by programmed recoding signals within the mRNA or at sites that are prone to frameshift errors.

Frameshifting has been suspected of participating in the generation of cryptic epitopes (Mayrand and Green, Immunol Today 19, no. 12:551, 1998; Elliott, et al, European Journal of Immunology 26:1175, 1996) but, the potential of this mechanism has never been directly tested. The invention disclosed herein shows the potential for frameshifting to be quite high. The mechanism is distinct from that proposed by Townsend and colleagues to produce unique tumor antigens, through the insertion and/or deletion of nucleotides in open reading frames, such as that encoding the adenometous polyposis coli (APC) gene, during the transformation process (Townsend, et al, Nature 371:662, 1994). In this case, the ribosome is guided to alternative open reading frames by such deletions/insertions, while following the rules of conventional translation.

Some frameshifting, such as that associated with HIV and AZ, occurs with high efficiency and has clearly evolved to regulate gene expression. In such cases it is not surprising that T_{CD8+} generally highly sensitive, detect epitopes whose expression is dependent upon frameshifting directed by the HIV and AZ elements. However, other frameshifting elements, such as that of the eor prone TK sequence and the AZ-Stop mutant, are much less efficient (Horsburgh, et al, Cell, 86:949, 1996; Matsufuji, et al, Cell, 80:51, 1995). Using the T-hybridoma β-galactosidase system, the Ova₂₅₇₋₂₆₄ epitope behind the TK-FS element was recognized at a low but significant level relative to in-frame controls (FIG. 4), whereas the AZ-Stop abrogated recognition using this assay (FIG. 5). However, using the standard ⁵¹Cr-release assay in vitro (FIG. 6) and two in vivo assays (FIG. 7 and 8), the low level epitope expression driven by the AZ-Stop frameshift construct was easily detectable. In these cases, T_{CD8+} recognition was significant but lower than that observed with the higher expression directed by the HIV and AZ frameshift windows, indicating a dependence of T_{CD8+} activation levels on the amount of epitope expression.

In the case of the wild-type and mutant TK element, frameshifting is directed by a simple slippery site, composed of a run of seven guanosine residues. One percent of the time wild-type,TK is translated, the ribosome shifts into the +1 reading frame and terminates translation 30 amino acids later (based on (Hwang, et al, Proc Natl Acad Sci USA 91, no. 12:5461, 1994). There is no known biological role for this truncated species. In fact, there may be no biological role, with the loss of a small percentage of functional protein, due to frameshifting being evolutionarily acceptable. This notion seems reasonable given the recent suggestion that perhaps 30% of newly synthesized proteins fail, for various reasons, to reach a fully mature state and instead are targeted for proteasome-dependent degradation (Schubert, et al, Nature 404, no. 6779:770, 2000).

Given that many different sequences may constitute error prone frameshift sites, “unintentional” ribosomal frameshifting may occur at a low level during translation of a variety of genes. Further, error prone frameshifting may be particularly pronounced in viral pathogens whose codon usage is shifted relative to its host. For example, HIV-1 has an unusual A rich codon bias which is markedly different from the one used by highly expressed human genes (Kypr and Mrazek, Nature 327, no. 6117:20, 1987; Kypr, et al, Biochim Biophys Acta 1009, no. 3:280, 1989; Sharp, Nature 324, no. 6093:114, 1986; van Hemert and Berkout, J Mol Evol 41, no. 2:132, 1995). As the abundance of isoaccepting tRNA species correlates with codon usages of highly expressed genes, it is likely that this mismatch negatively effects HIV gene expression. Decreased translation rates and frameshifting errors may be predicted due to slow decoding of rare codons (Gallant and Lindsley, biochem Soc Trans 21, no. 4:817, 1993; Gallant and Foley, University Park Press, Baltimore, Md. 615, 1980; Belcourt and Farabaugh, Cell 62, no. 2:239, 1990). In fact, it has been demonstrated that converting unfavorable HIV-1 codon bias in HIV genes to the one used by human genes results in enhanced translation efficiency (Haas, et al, Curr Biol, no. 3:315, 1996; Kotosopoulou, et al, J Virol 74, no. 10:4839, 2000; zur Megede, et al, J Virol 74, no. 6:2628, 2000). Truncated products of translational frameshifting may have no role in viral pathogenicity, but are very likely to contribute to the pool of defective ribosomal products (“DRIPs”,(Yewdell, et al, Journal of Immunology 157:1823, 1996), that are exploited by the immune system to detect intracellular invasion.

Of the mechanisms that have been demonstrated to drive cryptic epitope expression, the potential for frameshifting seems particularly high. Earlier, it was determined that correction of a frameshifting mutation within influenza NP (Fetten and E. Gilboa, Journal of Immunology 147:2697, 1991) is due to a low level secondary initiation of translation at an internal in-frame AUG downstream from the frameshift mutation (Bullock and I. C. Eisenlohr, Journal of Experimental Medicine 184:1319, 1996). Subsequently, it was determined that the potential for ribosomal initiation at internal start codons is dictated by the context of the primary AUG as predicted by the work of Kozak (Kozak, Annual Review of Cell Biology 8:197, 1992 ). Recently, Shastri and co-workers have revealed another variation of translation initiation that can lead to cryptic epitope expression (Malarkannan, et al, Immunity 10, no. 6:681, 1999). In this case translation commences at a non-AUG codon and results in a protein whose nascent N-terminus is occupied by a residue other than methionine. The potential for these aberrant initiation events, however, appears limited since the ability to initiate translation diminishes progressively after the message has been engaged (Kozak, Molecular and Cellular Biology 7:3438, 1987; Luukkonen, et al, Journal of Virology 69:4086, 1995). In contrast, frameshifting may occur anywhere within the ORF given an appropriate codon and sequence context.

Both alternative initiation mechanisms require that 8-10 amino acids be translated prior to termination for an MHC class I restricted epitope to be generated. This condition will not always be met, since some alternative open reading frames encode less than 8 amino acids. However, as little as a single amino acid need be translated after frameshifting in order for a cryptic epitope to have been generated. For example, most human class I-restricted epitopes possess a basic or aliphatic residue at the C-terminus. A protein might contain a potentially strong epitope in RFO but for the lack of such a residue at the C-terminus, a condition that frameshifting could rectify.

Natural examples of frameshift-dependent epitopes have not yet been observed, but it seems highly likely that they would be with sufficient scrutiny. Indeed, when the predicted wild-type TK frameshift product (including the 7 amino acids preceding and all 30 amino acids following the TK frameshift) is analyzed for potential class I-restricted epitopes (Parker, et al, Immunol 152, no. 1:163, 1994, Rammansee, et al, Immunogenetics 50, no. 3-4:213, 1999), several reasonable candidates binding to a variety of different class I molecules emerge. Current conventional means of mapping class I-restricted epitopes, principally the use of synthetic overlapping peptides representing the entire ORF, preclude the identification of epitopes in alternative reading frames. Further, as pointed out by Mayrand and Green (Mayrand and Green, Immunol Today 19, no. 12:551, 1998), most epitope mapping projects that prove to be less than straightforward are likely set aside unless the task is sufficiently significant, such as in the mapping of a potential tumor-specific epitope. Therefore, still unknown are the degree to which frameshift-dependent and other kinds of cryptic epitopes contribute to the generation of an immune response and the definition of “self”, and the extent to which they can be exploited in countering pathogens and tumor cells. Whatever their frequency, such epitopes are uniquely appropriate for certain applications.

Finally, the results indicate the utility of T cell recognition as an assay for the study of frameshifting. T_{CD8+} recognition assays are performed with intact cells and, indeed, with whole animals, providing highly sensitive readouts in both settings. In this respect, the HIV and antizyme frameshift windows are of particular interest. HIV frameshifting will be an important target for anti-viral therapies (Dinman, et al, Trends Biotechnol 16, no. 4:190, 1998; Irvine, et al, N Z Med J 111, no. 10^{68:222, 1998}). Normal Gag-Pol ratios, determined by frameshifting, have been shown to be critical for viral packaging (Felsenstein and Goff, J Virol 62, no. 6:2179, 1988). Compounds that increase or decrease frameshifting at the HIV frameshift window will significantly impair viral propagation.

Likewise, the AZ frameshifting window is of particular interest as a target for anti-cancer therapies. Several key findings couple increased polyamine levels, antizyme, and its target ornithine decarboxylase (ODC) to cellular transformation and cancer progression (Auvinen, et al, Nature 360, no. 6402:355, 1992; Clifford, et al, Cancer Res 55, no. 8:1680, 1995; Iwata, et al, Oncogene 18, no. 1:165, 1999; Meyskens and Gerner, Clin Cancer Res 5, no. 5:945, 1999; Moshier, et al, Cancer Res 53, no. 11:2618, 1993; Satriano, et al, J Biol Chem no. 25:15313; Tamori, et al, Cancer Res 55, no. 16:350, 1995). Recent efforts towards modulating polyamine levels for cancer intervention have used compounds such as the natural polyamine agmatine or polyamine analogues to increase antizyme expression via increased frameshifting. This strategy has the advantage of inhibiting both ODC, the rate limiting enzyme in polyamine biosynthesis, and polyamine transport through the action of increased antizyme levels. Peptide presentation-based readouts provide an excellent method of evaluating translational effects of potential anti-viral and anti-cancer compounds both in vitro and in vivo.

Alternatives and Extensions:

Several aspects of the present invention can be varied. While the present invention uses recombinant vaccinia technology to effect expression of the antigen, other methods are just as viable. These include, but are not limited to, injection of plasmid in which expression of the construct is driven by a eukaryotic promoter. This strategy has been shown by a large number of different groups to elicit antigen-specific T_{CD8+} responses. Other virus vectors could be used, including but not limited to, adenovirus or adeno-associated virus.

The present invention uses standard ⁵¹Cr-release and ELISPOT assays for measuring T_{CD8+} expansion, but another way to measure in vivo responses (Wherry, et al, J Immunol 163, No. 7:3735, 1999) is through the use of peptide-loaded MHC class I tetramers (Murali-Krishna et al., Immunity 8 177-187,1998). MHC/peptide complexes interact with T cell receptors with very low avidity. Multimerizing and labeling the ligand (MHC/peptide) with a fluorescent tag overcomes this shortcoming and allows the direct visualization of antigen-specific cells.

Finally, the level of the test construct expressed is varied so that drug-induced changes in recoding efficiency are more easily detected. This could be achieved by mutation of the promoter that drives transcription of the construct. Alternatively, this could be regulated at the level of translation.

The present invention includes a system for limiting, in a very controlled fashion, the amount of antigen that is expressed by inserting thermostable duplex structures (hairpins) between the promoter and the open reading frame of the gene under study. Such hairpins serve to impede the progression of the ribosome as it scans for the initiation codon. The larger the hairpin, the less frequently translation is achieved (Bullock and Eisenlohr, Journal of Experimental Medicine 184, 1319-1330, 1996; Wherry et al., J Immunol 163, 3735-45, 1999; Yellen-Shaw et al., Journal of Experimental Medicine 186, 1655-1662, 1997). These hairpin structures are used to reduce expression of the construct to a level that leads to submaxi T cell expansion, thereby allowing a more sensitive detection of changes in recoding in either the +1 or −1 direction.

1. A method for measuring efficacy of a compound to alter recoding of a translational reading frame, comprising:
a) constructing a nucleic acid cassette by inserting a recoding causing sequence upstream of a translational reading frame consisting of an MHC I restricted epitope encoding sequence, wherein said recoding causing sequence is placed in an alternative reading frame or beyond an upstream stop codon from that of said epitope encoding sequence so that recoding of said recoding causing sequence must take place in order for sai an MHC I restricted epitope to be expressed;
b) inserting said nucleic acid cassette of step a) into an expression vector;
c) infecting cells expressing an appropriate MHC class I molecule with said expression vector of step b);
d) applying a compound to said cells; and
e) determining efficacy of said compound to alter recoding of the recoding causing sequence by measuring activation of CD8+ T-cells specific for the epitope encoded by the epitope encoding sequence, wherein difference in activation of said T-cells compared to a control, wherein no compound has been added to the cells, indicates that the compound has capacity to alter recoding of the recoding causing sequence.

2. The method of claim 1, wherein the recoding causing sequence causes a −1 frameshifting event.

3. The method of claim 1, wherein the recoding causing sequence causes a +1 frameshifting.

4. The method of claim 1, wherein the recoding causing sequence causes a stop codon readthrough or a redefinition event.

5. The method of claim 1, wherein said recoding causing sequence comprises a sequence of a viral gene, wherein recoding of said recoding causing sequence results in translation of a protein.

6. A method of claim 1, wherein said recoding causing sequence comprises a gene sequence comprising a mutation resulting in a premature stop codon in a protein encoded by said gene sequence.

7. The method of claim 1, wherein said recoding causing sequence comprises a gene sequence encoding a protein influencing cell proliferation.

8. A method for measuring whether a test compound is capable of altering recoding of a translational reading frame, comprising:
a) constructing a nucleic acid cassette by inserting a nucleic acid sequence causing recoding upstream of an MHC I restricted epitope encoding nucleic acid sequence, wherein said sequence of causing recoding is placed in an alternative reading frame, or beyond an upstream stop codon, from that of said epitope encoding sequence so that recoding of said sequence must take place in order for said MHC I restricted epitope to be expressed;
b) inserting said nucleic acid cassette of step a) into an expression vector thereby allowing for expression of said MHC I restricted epitope in said expression vector;
c) infecting a mouse expressing an appropriate MHC class I molecule with said MHC I restricted epitope encoding sequence expressing vector of step b);
d) administering a test compound to said mouse;
e) expressing said MHC I restricted epitope in said mouse of step d); and
f) measuring an activation of epitope specific CD8⁺ T-cells, wherein change in the activation compared to CD8⁺ cells taken from a mouse not treated with the test compound indicates that said compound is capable of influencing altering recoding of a translational reading frame.

9. The method of claim 8, further comprising magnifying said MHC I restricted epitope specific CD8+ T-cells by restimulation in vitro with cells expressing said epitope.

10. The method of claim 8, comprising varying an amount of said test compound given to said mouse and measuring activation of the epitope specific CD8+ T-cells after administration of each amount of test compound to detect changes in recoding efficiency.

11. The method of claim 8, wherein the recoding causing sequence causes a −1 frameshifting event.

12. The method of claim 8, wherein the recoding causing sequence causes a +1 frameshifting event.

13. The method of claim 8, wherein the recoding causing sequence causes a stop codon readthrough or redefinition event

14. The method of claim 8 wherein said sequence causing recoding comprises a sequence in a viral gene wherein recoding of said sequence results in translation of a protein.

15. The method of claim 8 wherein said sequence causing recoding comprises a sequence in a gene wherein said sequence comprises a mutation in one nucleotide resulting in a premature stop codon in a gene encoding a protein, thereby causing a premature termination of said protein.

16. The method of claim 8 wherein said sequence causing recoding comprises a sequence in a gene encoding a protein influencing proliferation of a cell.

17. CANCELLED.

18. CANCELLED.

19. CANCELLED.

20. CANCELLED.

21. CANCELLED.

22. CANCELLED.

23. CANCELLED.

24. A method of identifying a recoding causing sequence, the method comprising the steps of:
a) constructing a nucleic acid cassette by inserting a sequence suspected of causing recoding upstream of a translational reading frame consisting of an MHC I restricted epitope encoding sequence, wherein said sequence suspected of causing recoding is placed in an alternative reading frame or beyond an upstream stop codon from that of said epitope encoding sequence so that recoding of said sequence suspected of causing recoding must take place in order for said epitope to be expressed;
b) inserting said nucleic acid cassette of step a) into an expression vector;
c) infecting cells expressing an appropriate MHC class I molecule with said expression vector of step b); and
e) measuring activation of CD8+ T-cells specific for the epitope encoded by the epitope encoding sequence, wherein activation of said CD8+ T-cells indicates identification of a recoding causing sequence.

25. The method of claim 23, wherein measuring activation is performed by measuring expansion of CD8+ T-cells.


SEQUENCE LISTING (10 sequences)

SEQ ID NO: 1 - DNA, 62 bp, Artificial Sequence
  synthetic oligonucleotides
cgctaattttttagggaagatctggccttcctacaagggaaggccagggaattttcttcatg

SEQ ID NO: 2 - DNA, 62 bp, Artificial Sequence
  synthetic oligonucleotides
cgctaattttctagggaagatctggccttcctacaagggaaggccagggaattttcttcatg

SEQ ID NO: 3 - DNA, 63 bp, Artificial Sequence
  synthetic oligonucleotides
cgctaattttttagggaagatctggccttcctacaagggaaggccagggaattttcttccatg

SEQ ID NO: 4 - DNA, 43 bp, Artificial Sequence
  synthetic oligonucleotides
cctggctcctcatatcggggggggaggctgggagctcagcatg

SEQ ID NO: 5 - DNA, 37 bp, Artificial Sequence
  synthetic oligonucleotides
cctggctcctcatatcggaggctgggagctcagcatg

SEQ ID NO: 6 - DNA, 42 bp, Artificial Sequence
  synthetic oligonucleotides
cctggctcctcatatcgggggggaggctgggagctcagcatg

SEQ ID NO: 7 - DNA, 79 bp, Artificial Sequence
  synthetic oligonucleotides
ctggtgctcctgatgtccctcacccacccctgaagatcccaggtgggcgagggaacagtcagcgggatcacagcgcatg

SEQ ID NO: 8 - DNA, 79 bp, Artificial Sequence
  synthetic oligonucleotides
ctggtgctccggatgtccctcacccacccctgaagatcccaggtgggagagggaacagtcagcgggatcacagcgcatg

SEQ ID NO: 9 - DNA, 80 bp, Artificial Sequence
  synthetic oligonucleotides
ctggtgctcctgatgtccctcacccacccctgaagatcccaggtgggcgagggaacagtcagcgggatcacagccgcatg

SEQ ID NO: 10 - DNA, 78 bp, Artificial Sequence
  synthetic oligonucleotides
ctggtgctccggatgtccctcacccacccctgaagatcccaggtgggcgagggaacagtcagcgggatcacaggcatg